(19) United States
(12) Patent Application Publication
(10) Pub. No.: US 2015/0292495 A1
(43) Pub. Date: Oct. 15, 2015

(54) SYRINGE QUICK DISCONNECT APPARATUS AND RELATED METHOD

(71) Applicant: Norgren Kloehn, Inc., Las Vegas, NV (US)

(72) Inventors: Brian Sweeney, Las Vegas, NV (US); Leonardo Diego Moral, Las Vegas, NV (US); Eriberto Miranda, Las Vegas, NV (US); Ian Kalani Castro, Las Vegas, NV (US); Dickson Ong, Las Vegas, NV (US); Gary Nielsen, Henderson, NV (US)

(73) Assignee: Norgren Kloehn, Inc., Las Vegas, NV (US)

(21) Appl. No.: 14/252,462

(22) Filed: Apr. 14, 2014

(51) Int. Cl. F04B 19/22; F04B 13/00; F04B 9/02

(52) U.S. Cl. CPC F04B 19/22; F04B 9/02; F04B 13/00; A61M 5/31533

(57) ABSTRACT

A fluid metering system having a syringe assembly attachable to a syringe dock is provided. The fluid metering system also comprises a plunger assembly with the syringe assembly that has a plunger configured to aspirate a fluid into the syringe assembly and/or dispense a fluid contained in the syringe assembly. A driving portion of a syringe drive with the fluid metering system is attachable to the plunger assembly, wherein the driving portion is configured to actuate dispensing and/or aspirating of the fluid. A slot with the syringe dock is configured to accept the syringe assembly, and an end cap with the syringe assembly slidingly engages the slot.

### FIELD OF THE INVENTION

The embodiments described below relate to liquid metering, and more particularly, to an improved syringe quick disconnect interface and related methods.

### BACKGROUND

Syringes generally include a plunger assembly that moves within a fluid bore. The plunger can draw in (aspirate) a fluid when the plunger is retracted from the bore and can dispense fluid when the plunger is pushed into the bore. Syringes are known for their precise fluid control and thus, have received great commercial success in the medical and laboratory fields. Typically, a syringe includes a single fluid port, wherein the fluid is drawn into and expelled from the bore through the port.

Although the syringes can be controlled manually, they are often utilized with electronic syringe pumps to provide an automated system. Such pump systems generally utilize a lead screw or ball screw driven by a stepper motor to effectuate precise control of the syringe plunger's position. In the medical field, syringe pumps can provide, for example, automated dosing to a patient, and in laboratory settings they may be used for precise reagent metering. In scientific/industrial settings, such pumps are often employed in chromatography, electrophoresis, hematology, flow cytometry, immunoassays, and even for refilling ink cartridges.

In some situations, the syringe pump can form part of a larger syringe pump manifold system wherein multiple syringes are coupled to the manifold system. Each syringe may include its own pump or a single syringe pump may control multiple syringes. The syringe pump may control the flow of fluid into or out of a syringe. The syringe pump may, in addition, combine fluids from more than one syringe.

In order to provide a fluid-tight coupling between the syringe and the manifold assembly, a separate sealing member is often required. Positioning the separate sealing member while simultaneously attaching the syringe to the manifold assembly is generally difficult. One source of difficulty stems from the syringe being typically inserted into a bottom surface of the manifold or some other syringe accepting port. Consequently, there is no place for the sealing member to rest before coupling the syringe to its corresponding port. Therefore, a user generally attempts to hold the sealing member in place with one hand while coupling the syringe to the manifold with the other. This makes for a difficult task especially if the syringe being coupled is positioned between two other adjacent syringes, resulting in limited space for a user to maneuver.

In other attachment schemes, a Luer fitting is often utilized for fluid connections. Luer connectors (often marked by the name “Luer-Lock”) are used for making leak-free connections between a male-taper fitting of the syringe itself and its mating female portion found on medical and laboratory instruments, such as hypodermic syringe tips/needles or stopcocks. A Luer-Lock requires that the user thread the syringe onto the mating portion. This typically takes two hands to accomplish, and also requires a reasonable amount of space between syringes on a manifold for finger clearance during threading. Similarly, interfaces with ¼-28 UNF-2A threaded ends have the same limitations. Luer-lock fittings requires about half a turn and ¼-28 UNF-2A fittings require up to five turns.

Overall, the assembly process—inserting and removing syringes to and from manifolds/pumps—becomes increasingly difficult with multi-channel units. Often, it is necessary that a syringe be replaced or taken off the pump, but because of the pump's compact design, it is very difficult to remove a syringe that is in the middle of neighboring syringes, as there is insufficient space for a person's fingers to provide clearance so to maintain a proper grip. It is also difficult or at least ill-advised to use a pair of pliers to aid in syringe installation and removal. Besides risking breakage of a syringe, either not enough torque may be applied onto the syringe assembly or too much torque may be applied.

Therefore, there is a need for an apparatus that can simultaneously hold onto the sealing member and the syringe to create a fluid tight junction. Therefore, there is a need for an apparatus that can simultaneously hold onto the sealing member and the syringe to create a fluid tight junction without the need to rotate the syringe for threading. There is a need to alleviate the difficulty of removing a syringe that is bordered by two other syringes when installed on a manifold or pump. There is a need to alleviate the difficulty of re-attaching a syringe onto a pump/manifold. There is a need to provide an apparatus and method to aid in the assembly and dissasembly of syringes into manifolds/pump while providing the proper installation force. There is a need to minimize glass syringe barrel breakage by eliminating the need for users to grab the glass barrel in order to secure to or remove the syringe from the pump valve and/or manifold (when this is done, the glass barrel can break off from the end cap). There is a need for a device that can flex to allow a user to pull the device away from the syringe once the syringe is at least partially coupled to the manifold in order to remove the apparatus without damaging the syringe or removing the sealing member.

The embodiments described below overcome these and other problems and an advance in the art is achieved. The embodiments described below provide an apparatus that can retain a sealing member against a syringe while the syringe is being coupled to a manifold or some other type of port. This allows a user to move the syringe into position to be coupled to the manifold easily. The apparatus further includes deformable members that accommodates a syringe while allowing a user to pull a syringe directly away from the apparatus. Similarly, the apparatus provides the ability to simply press a syringe into place for installation into a pump or manifold.

### SUMMARY OF THE INVENTION

A fluid metering system having a syringe assembly attachable to a syringe dock is provided according to an embodiment. According to an embodiment a plunger assembly with the syringe assembly has a plunger configured to at least one of aspirate a fluid into the syringe assembly and dispense a fluid contained in the syringe assembly. A driving portion of a syringe drive with the fluid metering system is attachable to the plunger assembly, wherein the driving portion is configured to actuate at least one of dispensing and aspirating of the fluid. A slot with the syringe dock is configured to accept the syringe assembly, and an end cap with the syringe assembly slidingly engages the slot.

A fluid metering system having a syringe assembly attachable to a polymer syringe dock is provided according to an embodiment. According to an embodiment a plunger assembly with the syringe assembly has a plunger configured to at least one of aspirate a fluid into the syringe assembly and dispense a fluid contained in the syringe assembly. A driving portion of a syringe drive with the fluid metering system is attachable to the plunger assembly, wherein the driving portion is configured to actuate at least one of dispensing and aspirating of the fluid. A slot with the syringe dock is configured to accept the syringe assembly. At least one detent with the slot is configured to engage at least one member of the end cap. A polymer end cap with the syringe assembly slidingly engages the slot. A compliant seal is overmolded with the end cap proximate an orifice that passes through the end cap, wherein the seal is configured to fluidly seal the end cap to the syringe dock such that the orifice is in sealed fluid communication with the fluid metering system, wherein an insertion force necessary for the end cap to engage the slot is between approximately 2.5 and approximately 4.5 pounds.

A method of using a fluid metering system having a syringe assembly attachable to a syringe dock is provided according to an embodiment. According to an embodiment the method comprises the steps of: sliding the syringe assembly into the syringe dock such that the syringe assembly engages the syringe dock and fluidly connects the syringe assembly with the fluid metering system; and attaching the plunger assembly to the syringe drive.

### ASPECTS

According to an aspect, a fluid metering system having a syringe assembly attachable to a syringe dock is provided. According to an embodiment:
a plunger assembly with the syringe assembly has a plunger configured to at least one of aspirate a fluid into the syringe assembly and dispense a fluid contained in the syringe assembly;
a driving portion of a syringe drive with the fluid metering system is attachable to the plunger assembly, wherein the driving portion is configured to actuate at least one of dispensing and aspirating of the fluid;
a slot with the syringe dock is configured to accept the syringe assembly; and
an end cap with the syringe assembly slidingly engages the slot.

Preferably, the end cap comprises PTFE.

Preferably, the syringe dock comprises PEEK.

Preferably, the plunger assembly is attachable to the driving portion of the syringe drive by a magnetic force.

Preferably, the fluid metering system further comprises:
a through hole with the plunger assembly; and
a boss defined by the driving portion of the syringe drive configured to pass through the through hole and actuate the plunger assembly.

Preferably, the fluid metering system further comprises:
a seal with the end cap proximate an orifice passing through the end cap, wherein the seal is configured to fluidly seal the end cap to the syringe dock such that the orifice is in sealed fluid communication with the fluid metering system.

Preferably, the seal comprises perfluoroelastomer.

Preferably, the seal is overmolded with the end cap.

Preferably, the seal is an o-ring.

Preferably, the fluid metering system further comprises at least one detent with the slot configured to engage at least one member, of the end cap.

Preferably, an insertion force necessary for the end cap to engage the slot is between approximately 2.5 and approximately 4.5 pounds.

Preferably, an insertion force necessary for the end cap to engage the slot is approximately 3.4 pounds.

Preferably, the end cap comprises:
a first member proximate a second end of the end cap;
a second member proximate a fist end of the end cap; and
an intermediary region of the end cap disposed between the first member and the second member.

Preferably, the fluid metering system further comprises:
a first diameter of the first member is greater than a diameter of the intermediary region; and
a second diameter of the second member is greater than the diameter of the intermediary region.

Preferably, the fluid metering system further comprises a radiused ramp that connects the intermediary region to the first member.

According to an aspect, a fluid metering system having a syringe assembly attachable to a polymer syringe dock is provided. According to an embodiment:
a plunger assembly with the syringe assembly has a plunger configured to at least one of aspirate a fluid into the syringe assembly and dispense a fluid contained in the syringe assembly;
a driving portion of a syringe drive with the fluid metering system is attachable to the plunger assembly, wherein the driving portion is configured to actuate at least one of dispensing and aspirating of the fluid;
a slot with the syringe dock is configured to accept the syringe assembly;
at least one detent with the slot is configured to engage at least one member, of the end cap;
a polymer end cap with the syringe assembly slidingly engages the slot;
a compliant seal overmolded with the end cap is proximate an orifice passing through the end cap, wherein the seal is configured to fluidly seal the end cap to the syringe dock such that the orifice is in sealed fluid communication with the fluid metering system, wherein an insertion force necessary for the end cap to engage the slot is between approximately 2.5 and approximately 4.5 pounds.

Preferably, an insertion force necessary for the end cap to engage the slot is approximately 3.4 pounds.

Preferably, the end cap comprises:
a first member proximate a second end of the end cap;
a second member proximate a fist end of the end cap; and
an intermediary region of the end cap disposed between the first member and the second member.

Preferably, the fluid metering system has a first diameter of the first member that is greater than a diameter of the intermediary region; and a second diameter of the second member that is greater than the diameter of the intermediary region.

According to an aspect, a method of using a fluid metering system having a syringe assembly attachable to a syringe dock is provided. The method comprises the steps of:
sliding the syringe assembly into the syringe dock such that the syringe assembly engages the syringe dock and fluidly connects the syringe assembly with the fluid metering system; and attaching the plunger assembly to the syringe drive.

Preferably, the plunger assembly is configured to at least one of aspirate a fluid into the syringe assembly and dispense a fluid contained in the syringe assembly, and wherein the syringe drive is configured actuate the plunger assembly to at least one of dispense and aspirate a fluid.

Preferably, the step of sliding the syringe assembly into the syringe dock comprises fluidly connecting the syringe assembly with the fluid metering system and attaching the plunger assembly to the syringe drive substantially simultaneously.

Preferably, the syringe dock comprises a slot configured to accept the syringe assembly; an end cap with the syringe assembly is configured to slidingly engage the slot; and wherein the step of pressing the syringe assembly into the syringe dock comprises the step of engaging a detent of the syringe dock with the end cap.

Preferably, the step of attaching the plunger assembly to the syringe drive comprises magnetically attaching the plunger assembly to the syringe drive.

Preferably, the step of attaching the plunger assembly to the syringe drive comprises passing a boss with the driving portion of the syringe drive through a through hole with the plunger assembly.

Preferably, the step of sliding the syringe assembly into the syringe dock comprises encountering an insertion force resistance between approximately 2.5 and approximately 4.5 pounds

Preferably, the step of sliding the syringe assembly into the syringe dock comprises encountering an insertion force resistance is approximately 3.4 pounds.

Preferably, the end cap comprises:
a first member proximate a second end of the end cap;
a second member proximate a fist end of the end cap; and
an intermediary region of the end cap disposed between the first member and the second member.

Preferably, the end cap comprises:
a first member proximate a second end of the end cap;
a second member proximate a fist end of the end cap; and
an intermediary region of the end cap disposed between the first member and the second member.

Preferably, a first diameter of the first member is greater than a diameter of the intermediary region; and a second diameter of the second member is greater than the diameter of the intermediary region.

### BRIEF DESCRIPTION OF THE DRAWINGS

The same reference number represents the same element on all drawings. The drawings are not necessarily to scale.

FIG. 1 illustrates a syringe assembly according to an embodiment;

FIG. 2A illustrates a fluid metering system according to an embodiment;

FIG. 2B illustrates the fluid metering system of FIG. 2A;

FIG. 2C illustrates a fluid metering system of FIGS. 2A-B with a syringe assembly attached thereto;

FIG. 3A illustrates a fluid metering system according to an embodiment;

FIG. 3B illustrates the fluid metering system of FIG. 3A;

FIG. 4A illustrates an isometric view of an end cap according to an embodiment;

FIG. 4B illustrates a side view of the end cap of FIG. 4A;

FIG. 4C illustrates a side cross-sectional view of the end cap of FIGS. 5A-B;

FIG. 5A illustrates an isometric view of an end cap according to an embodiment;

FIG. 5B illustrates a side view of the end cap of FIG. 5A;

FIG. 5C illustrates a side cross-sectional view of the end cap of FIGS. 5A-B;

FIG. 6A illustrates a side cross-sectional view of a syringe dock according to an embodiment;

FIG. 6B illustrates a magnified view of the side cross-sectional view of the syringe dock of FIG. 6A;

FIG. 6C illustrates a magnified front view of the of the syringe dock of FIGS. 6A-B; and

FIG. 6D illustrates a magnified isometric view of the of the syringe dock of FIGS. 6A-C.

### DETAILED DESCRIPTION OF THE INVENTION

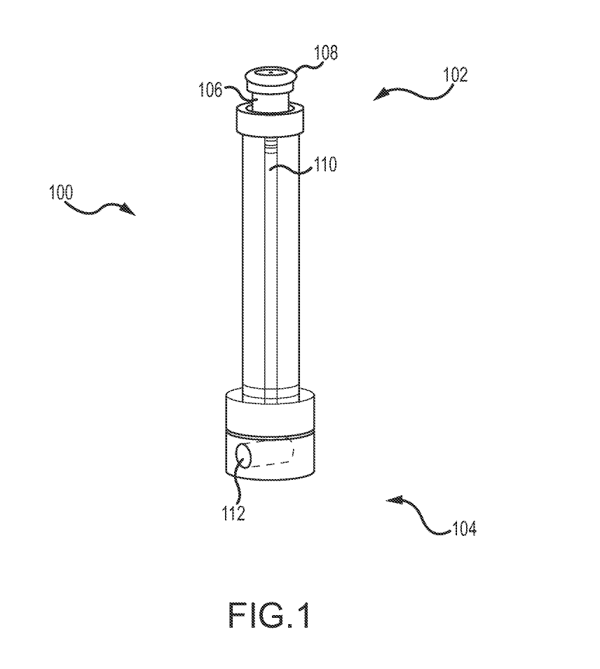

FIGS. 1-6D and the following description depict specific examples to teach those skilled in the art how to make and use the best mode of embodiments of a fluid metering system and related methods. For the purpose of teaching inventive principles, some conventional aspects have been simplified or omitted. Those skilled in the art will appreciate variations from these examples that fall within the scope of the invention. Those skilled in the art will appreciate that the features described below can be combined in various ways to form multiple variations of the invention. As a result, the invention is not limited to the specific examples described below, but only by the claims and their equivalents.

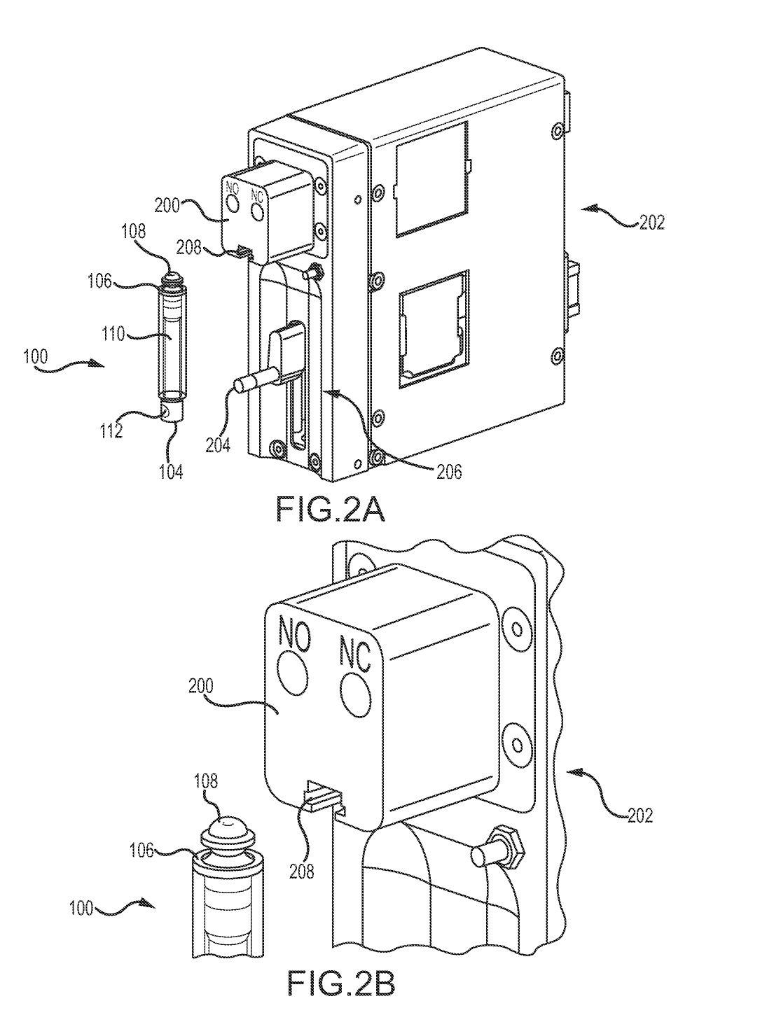

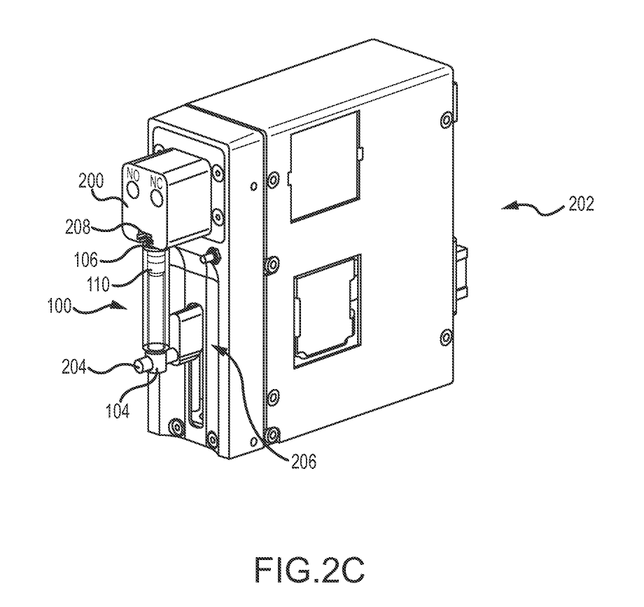

With initial reference to FIG. 1, a syringe assembly 100 is constructed from two main sub-assemblies: a barrel assembly 102 and a plunger assembly 104. The barrel assembly 102 provides means to mount the syringe assembly 100 to a syringe dock 200 (see FIG. 2), to, in one embodiment, fluidly connect the syringe assembly 100 to a fluid metering system 202, such as a valve and/or manifold assembly (see FIG. 2).

Turning to FIGS. 2A-C, an end cap 106 of the barrel assembly 102 allows attachment of the syringe assembly 100 to the syringe dock 200 to be a simple push-on/quick disconnect process. The plunger assembly 104 attaches to a driving portion 204 of the syringe drive 206, which actuates the plunger assembly 104 to aspirate and dispense fluid located into/from the syringe assembly 100 from/to the fluid metering system 202. The syringe drive 206 utilizes a lead screw, ball screw, linear actuator, or any other drive known in the art, and is preferably driven by a stepper motor to effectuate precise control of the syringe plunger's 110 position. A stepper motor serves as a non-limiting example, for other drive mechanisms besides stepper motors are also contemplated. The syringe drive 206 is controlled by at least one of a microprocessor, computing device, and electronics components (not shown), as will be understood by one skilled in the art.

By way of example, a user would, as illustrated in FIGS. 2A-2B, place the syringe assembly 100 proximate the fluid metering system 202, aligning the end cap 106 with a slot 208 defined by the syringe dock 200. Also, the opposite end of the syringe assembly 100 is oriented such that the driving portion 204 of the syringe drive 206 is aligned with a portion of the plunger assembly 104. In one embodiment (as illustrated in FIGS. 2A-C), a through hole 112 of the plunger assembly 104 is configured to engage a rod defined by the driving portion 204. By holding the syringe assembly 100 as described, simply sliding the end cap 106 into the slot 208 of the syringe dock 200 and allowing the rod defined by the driving portion 204 to pass through the through hole 112, the syringe assembly 100 is securely held in place by the fluid metering system 202, the seal 108 engages the syringe dock 200 to create a fluid tight junction, and the syringe plunger 110 is drivable by the driving portion 204. This is all accomplished without the need to rotate the syringe assembly 100 for threading.

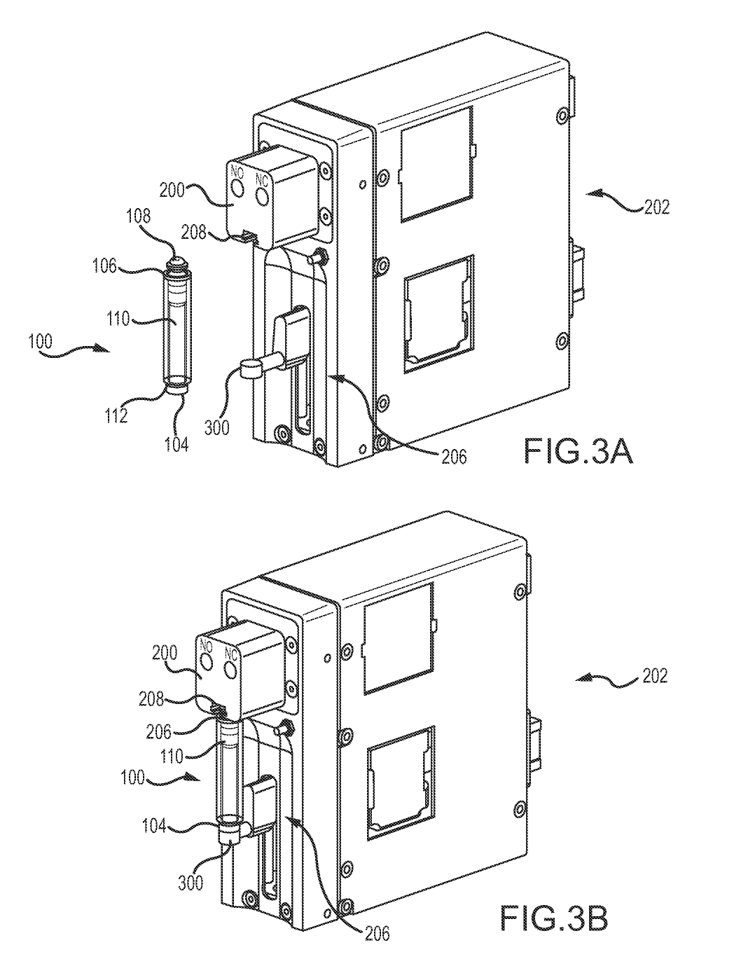

In an alternate embodiment illustrated by FIGS. 3A-B, a magnet 300 with the driving portion 204 engages the plunger assembly 104. In this embodiment, the plunger assembly comprises a ferrous or magnetic portion 302 that is attracted to the magnet 300. It will be apparent to one skilled in the art that the magnet 300 can be substituted with a ferrous material, and the plunger assembly 104 may comprise a magnet. This magnetic coupling allows the syringe drive 206 to drive the plunger assembly 104.

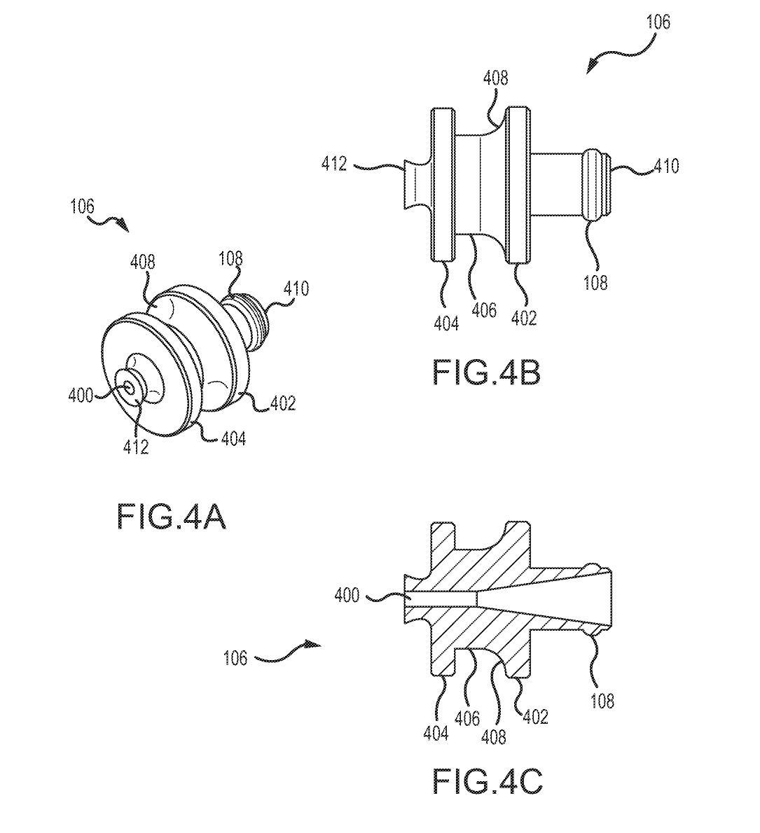

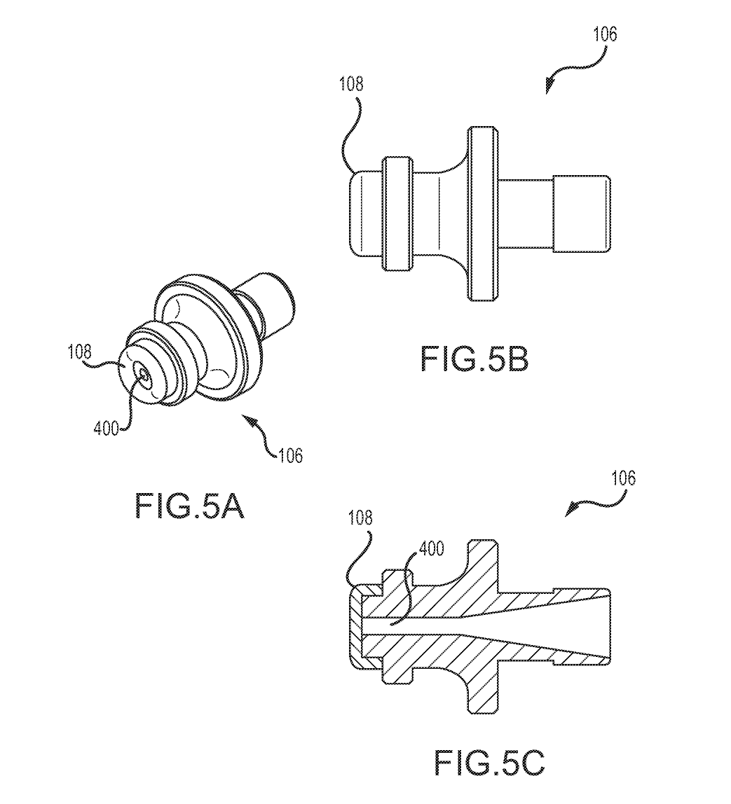

With reference to FIGS. 1, 2A-C, and now 4A-C, The seal 108 of the end cap 106 provides a fluid tight seal when the syringe assembly 100 is docked to the syringe dock 200. When docked, fluid in the syringe assembly 100 may flow to the fluid metering system 202 through an orifice 400 of the end cap 106. The seal 108 is constructed from a compliant material. Materials contemplated are, for example without limitation, butadiene rubber, butyl rubber, chlorosulfonated polyethylene, epichlorohydrin rubber, ethylene propylene diene monomer, ethylene propylene rubber, fluroelasomer, nitrile, perflurorelastomer, polyacrylate rubber, polychloroprene, polyisoprene, polysulfide rubber, polytetrafluoroethylene, silicone rubber, styrene butadiene rubber, thermoplastic elastomer, thermoplastic polyolefin, thermoplastic polyurethane, thermoplastic etheresterelastomers, thermoplastic polyamide, and any other material known in the art. In one embodiment, an O-ring or similar seal is provided with the end cap 106 to aid in sealing the syringe assembly 100 to the syringe dock 200. In another embodiment, a compliant material is over-molded on a base material. In a preferred embodiment (see FIGS. 4A-C), the base material of the end cap 106 comprises polyether ether ketone (PEEK) with an over-molded seal 108 made from perfluoroelastomer (FFKM). In another preferred embodiment (see FIGS. 5A-C), the base material of the end cap 106 comprises polytetrafluoroethylene (PTFE) with an over-molded seal 108 made from perfluoroelastomer (FFKM). Other polymers for the end cap 106 contemplated are perfluoroalkoxy, fluorinated ethylene propylene, high-density polyethylene, metals, ceramics, plastics, and any other material known in the art.

The precision design of the end cap 106 contours shown in FIGS. 4A-C and 5A-C are calculated theoretically. The end cap 106 to slot 208 interface is calculated as a function of the material properties making contact with each other. The equations take into account the material properties such as the coefficient of friction and the elastic modulus, which provide precise dimensions for the end cap 106, seal 108 and the slot 208 needed for an effective insertion force for stable assembly and maintenance of the syringe 100 onto the fluid metering system 202. The precise design of the contours yields a tactile detent feedback indicating to a user that the syringe 100 is securely in place and that the orifice 400 is in fluid-tight communication with fluid metering system 202.

FIGS. 4A-C illustrate the end cap 106 having a first member 402 having a radius, a second member 404 having a radius, and an intermediary region 406 having a radius. A ramp 408 connects the intermediary region 406 with the first member 402. The orifice 400 passes through the entire end cap 106 from a first end 410 to a second end 412. The overmolded seal 108 is proximate the first end 410. The spacing between the first and second members 402, 404 as well as their respective radii, in combination with the radius of the intermediary region 406 and the radius of the ramp 408—in addition to other dimensions—create a preferred fit between the end cap 106 and the dimensions of the syringe dock 200 slot 208.

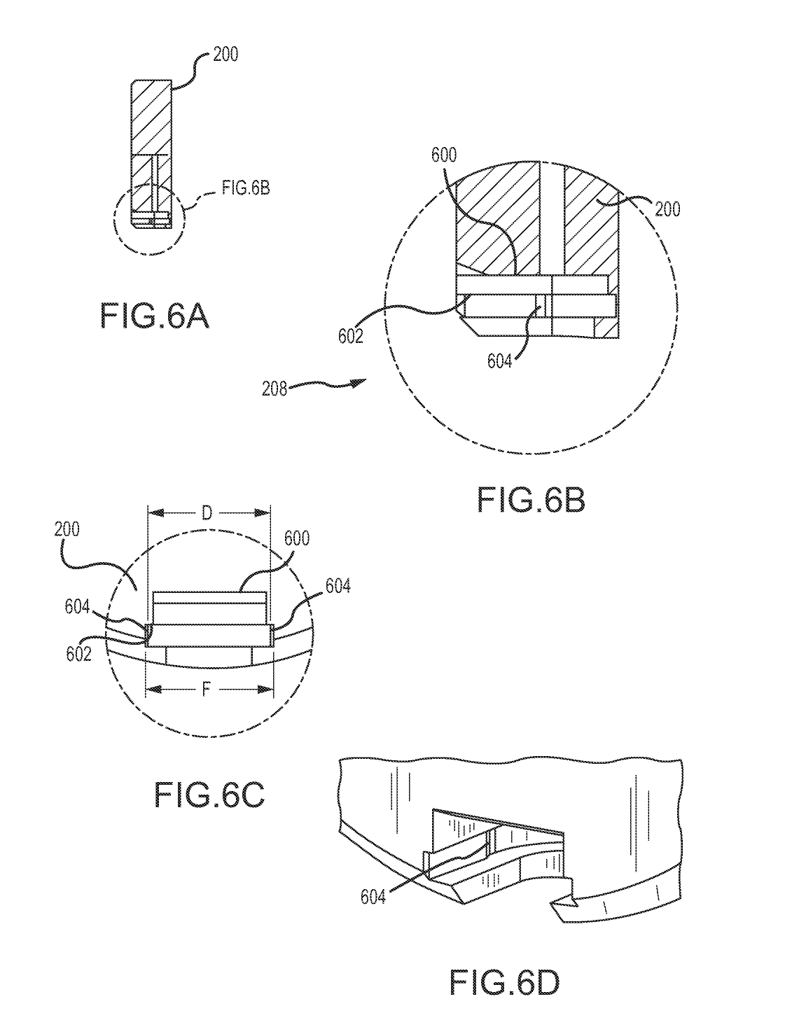

In an example of a calculation used to design an embodiment of the end cap 106, compressive forces F_{comp1}, F_{comp2} and F_{comp3} are calculated (see below). With reference to FIGS. 6A-D, as the end cap 106 is inserted into the slot 208, the seal 108 will be compressed where a top side of the seal 108 slides along the seal-bearing surface 600 of the slot 208 in the dock 200. The sliding action will cause the end cap 106 to encounter a frictional force between the seal 108 and the dock 200. The compression force imparted on the seal's 108 top surface is denoted as F_{comp1}. This, and the other compression forces, are determined using the criterion that the syringe assembly 100 undergoes 150 psi built-up pressure, for example, as it is actuated via the pump system with fluids flowing therethrough. These compression forces must be high enough so that the syringe to valve/manifold interface will not move under load or create a fluidic pathway where a leak will occur.

Equation number 1 is used to calculate frictional force:

F_{friction}=μ*F_{comp} (1)

Where:

F_{friction}=frictional force

μ=coefficient of friction

F_{comp}=compressive force

The example materials analyzed comprise Teflon PTFE, FKM, and PEEK. The static coefficient of friction is 0.24 for FKM and 0.14 for PTFE and PEEK.

### Therefore,

F_{friction1}=μ*F_{comp1}

F_{friction1}=0.24*10 pounds=2.4 pounds

Similarly, the equation for calculating the frictional force due to the Teflon first member 402 engaging the first member bearing surface 602 of the slot 208 as the first member 402 engages the PEEK dock 200 ledge is:

F_{friction2}=μ*F_{comp2}

F_{friction2}=0.14*10 pounds=1.4 pounds

The equation for calculating the frictional force due to the Teflon second member 404 along its outermost diameter to the PEEK dock's 200 detent 604 is:

F_{friction3}=μ*F_{comp3}

F_{friction3}=0.14*6.5 pounds=0.91 pounds

These are merely examples of calculations performed for an embodiment, and in no way should limit the scope of claims or this specification. In another example, more complex Monte Carlo analyses may be performed to aid in defining the appropriate dimensions for both the end cap 106 and slot 208 (and their various members, contours, ramps, undercuts, bosses, detents, etc.). In an example, the modulus of PEEK and PTFE as well as the coefficient of friction for FKM and PEEK/PTFE are known constants. Manufacturing tolerances and run-outs are known, and the dimensions of all portions of the both the end cap 106 and slot 208 are known.

Intermediate calculations, such as those noted for stress-strain (below) and frictional force (above), may be employed to calculate seal 108 stretch, detent 604 compression friction force, post-stretch seal 108 thickness, general compression forces, seal 108 friction force, and slot 208 friction forces. Another equation (Equation number 2, below) may be used to calculate stress or strain, for example:

σ=E*ε (2)

Where:

σ=stress

ε=strain

E=Elastic modulus (psi)

The Elastic modulus is known for materials used, such as for PEEK or PTFE:

E_{PEEK}=500,000 psi

E_{PTFE}=87,000 psi

For example, this equation may be used to determine the stress, σ, imparted, on the Teflon body.

Finally, Monte Carlo analysis may yield the theoretical insertion force for the end cap 106 into the slot 208 based upon the above inputted variables and intermediate calculations. Additionally, compression, clearance, seal 108 volume, orifice 400 runout, and front-to-front and side-to-side float of the end cap 106 while installed in the slot 208 may also be calculated. In one embodiment, the insertion force is under 10 lbs. In one embodiment, the insertion force is between 1.5 and 5.5 lbs. In another embodiment, the insertion force is between 2.5 and 4.5 lbs. In a preferred embodiment, the insertion force is approximately 3.4 lbs. These ranges are examples, and do not serve to limit the scope of the claims in any way.

It will be clear to one skilled in the art that adjusting the dimensions of at least one of the first member 402, second member 404, intermediary region 406, ramp 408, first end 410, and/or second end 412, as well as adjusting the material from which the end cap 106 is made will change the insertion force. Similarly, adjusting the dimensions of at least one of the seal-bearing surface 600, first member bearing surface 602, the slot 208 in general, and/or the detent 604 as well as adjusting the material from which the syringe dock 200 is made will change the insertion force.

The detailed descriptions of the above embodiments are not exhaustive descriptions of all embodiments contemplated by the inventors to be within the scope of the invention. Indeed, persons skilled in the art will recognize that certain elements of the above-described embodiments may variously be combined or eliminated to create further embodiments, and such further embodiments fall within the scope and teachings of the invention. It will also be apparent to those of ordinary skill in the art that the above-described embodiments may be combined in whole or in part to create additional embodiments within the scope and teachings of the invention.

Thus, although specific embodiments of, and examples for, the invention are described herein for illustrative purposes, various equivalent modifications are possible within the scope of the invention, as those skilled in the relevant art will recognize. The teachings provided herein can be applied to other devices and method, and not just to the embodiments described above and shown in the accompanying figures. Accordingly, the scope of the invention should be determined from the following claims.

What is claimed is:

1. A fluid metering system having a syringe assembly attachable to a syringe dock, comprising:
a plunger assembly with the syringe assembly having a plunger configured to at least one of aspirate a fluid into the syringe assembly and dispense a fluid contained in the syringe assembly;
a driving portion of a syringe drive with the fluid metering system attachable to the plunger assembly, wherein the driving portion is configured to actuate at least one of dispensing and aspirating of the fluid;
a slot with the syringe dock configured to accept the syringe assembly; and
an end cap with the syringe assembly that slidingly engages the slot.

2. The fluid metering system of claim 1, wherein the end cap comprises PTFE.

3. The fluid metering system of claim 1, wherein the syringe dock comprises PEEK.

4. The fluid metering system of claim 1, wherein the plunger assembly is attachable to the driving portion of the syringe drive by a magnetic force.

5. The fluid metering system of claim 1, further comprising:
a through hole with the plunger assembly; and
a boss defined by the driving portion of the syringe drive configured to pass through the through hole and actuate the plunger assembly.

6. The fluid metering system of claim 1, further comprising a seal with the end cap proximate an orifice passing through the end cap, wherein the seal is configured to fluidly seal the end cap to the syringe dock such that the orifice is in sealed fluid communication with the fluid metering system.

7. The fluid metering system of claim 6, wherein the seal comprises perfluoroelastomer.

8. The fluid metering system of claim 6, wherein the seal is overmolded with the end cap.

9. The fluid metering system of claim 6, wherein the seal is an o-ring.

10. The fluid metering system of claim 1, further comprising at least one detent with the slot configured to engage at least one member, of the end cap.

11. The fluid metering system of claim 1, wherein an insertion force necessary for the end cap to engage the slot is between approximately 2.5 and approximately 4.5 pounds.

12. The fluid metering system of claim 1, wherein an insertion force necessary for the end cap to engage the slot is approximately 3.4 pounds.

13. The fluid metering system of claim 1, wherein the end cap comprises:
a first member proximate a second end of the end cap;
a second member proximate a fist end of the end cap; and
an intermediary region of the end cap disposed between the first member and the second member.

14. The fluid metering system of claim 13, wherein:
a first diameter of the first member is greater than a diameter of the intermediary region; and
a second diameter of the second member is greater than the diameter of the intermediary region.

15. The fluid metering system of claim 13, further comprising a radiused ramp that connects the intermediary region to the first member.

16. A fluid metering system having a syringe assembly attachable to a polymer syringe dock, comprising:
a plunger assembly with the syringe assembly having a plunger configured to at least one of aspirate a fluid into the syringe assembly and dispense a fluid contained in the syringe assembly;
a driving portion of a syringe drive with the fluid metering system attachable to the plunger assembly, wherein the driving portion is configured to actuate at least one of dispensing and aspirating of the fluid;
a slot with the syringe dock configured to accept the syringe assembly;
at least one detent with the slot configured to engage at least one member, of the end cap;
a polymer end cap with the syringe assembly that slidingly engages the slot; and
a compliant seal overmolded with the end cap proximate an orifice passing through the end cap, wherein the seal is configured to fluidly seal the end cap to the syringe dock such that the orifice is in sealed fluid communication with the fluid metering system, wherein an insertion force necessary for the end cap to engage the slot is between approximately 2.5 and approximately 4.5 pounds.

17. The fluid metering system of claim 16, wherein an insertion force necessary for the end cap to engage the slot is approximately 3.4 pounds.

18. The fluid metering system of claim 16, wherein the end cap comprises:
a first member proximate a second end of the end cap;
a second member proximate a fist end of the end cap; and
an intermediary region of the end cap disposed between the first member and the second member.

19. The fluid metering system of claim 18, wherein:
a first diameter of the first member is greater than a diameter of the intermediary region; and
a second diameter of the second member is greater than the diameter of the intermediary region.

20. A method of using a fluid metering system having a syringe assembly attachable to a syringe dock, comprising the steps of:
sliding the syringe assembly into the syringe dock such that the syringe assembly engages the syringe dock and fluidly connects the syringe assembly with the fluid metering system; and
attaching the plunger assembly to the syringe drive.

21. The method of using a fluid metering system of claim 20, wherein the plunger assembly is configured to at least one of aspirate a fluid into the syringe assembly and dispense a fluid contained in the syringe assembly, and wherein the syringe drive is configured actuate the plunger assembly to at least one of dispense and aspirate a fluid.

22. The method of using a fluid metering system of claim 20, wherein the step of sliding the syringe assembly into the syringe dock comprises fluidly connecting the syringe assembly with the fluid metering system and attaching the plunger assembly to the syringe drive substantially simultaneously.

23. The method of using a fluid metering system of claim 20, wherein:
the syringe dock comprises a slot configured to accept the syringe assembly;
an end cap with the syringe assembly is configured to slidingly engage the slot; and
wherein the step of pressing the syringe assembly into the syringe dock comprises the step of engaging a detent of the syringe dock with the end cap.

24. The method of using a fluid metering system of claim 20, wherein the step of attaching the plunger assembly to the syringe drive comprises magnetically attaching the plunger assembly to the syringe drive.

25. The method of using a fluid metering system of claim 20, wherein the step of attaching the plunger assembly to the syringe drive comprises passing a boss with the driving portion of the syringe drive through a through hole with the plunger assembly.

26. The method of using a fluid metering system of claim 20, wherein the step of sliding the syringe assembly into the syringe dock comprises encountering an insertion force resistance between approximately 2.5 and approximately 4.5 pounds.

27. The method of using a fluid metering system of claim 20, wherein the step of sliding the syringe assembly into the syringe dock comprises encountering an insertion force resistance is approximately 3.4 pounds.

28. The method of using a fluid metering system of claim 23, wherein the end cap comprises:
a first member proximate a second end of the end cap;
a second member proximate a fist end of the end cap; and
an intermediary region of the end cap disposed between the first member and the second member.

29. The method of using a fluid metering system of claim 23, wherein the end cap comprises:
a first member proximate a second end of the end cap;
a second member proximate a fist end of the end cap; and
an intermediary region of the end cap disposed between the first member and the second member.

30. The method of using a fluid metering system of claim 29, wherein:
a first diameter of the first member is greater than a diameter of the intermediary region; and
a second diameter of the second member is greater than the diameter of the intermediary region.
